(19) United States
(12) Patent Application Publication
(10) Pub. No.: US 2022/0254456 A1
(43) Pub. Date: Aug. 11, 2022

(54) METHOD OF TRAINING A MODEL FOR DETERMINING A MATERIAL PARAMETER

(71) Applicant: Robert Bosch GmbH, Stuttgart (DE)

(72) Inventors: Dominic Lingenfelser, Hambruecken (DE); Elisabeth Lotter, Pforzheim (DE); Martin Beyer, Marbach (DE)

(21) Appl. No.: 17/626,048

(22) PCT Filed: Aug. 14, 2020
(86) PCT No.: PCT/EP2020/072854

§ 371 Date: Jan. 10, 2022

(30) Foreign Application Priority Data
Aug. 19, 2019 (DE) 10 2019 212 330.9

(51) Int. Cl. G16C 60/00; G16C 20/70

(52) U.S. Cl. CPC G16C 60/00; G16C 20/90; G16C 20/70

(57) ABSTRACT

A device and method for determining a material parameter, in particular, for a plastic material or a process. A combination of input variables for a model is provided. The material parameter is determined as a function of the model. The model maps the combination of input variables to material parameters. The model is trained as a function of training data, which are defined by a plurality of combinations of input variables and their specific assignment to a setpoint material parameter. Either the model continues to be trained as a function of a result of a comparison of a material parameter determined by the model for one of the combinations from the training data, with the setpoint material parameter assigned to this combination in the training data, or a changed model is defined, and the changed model is trained.

### FIELD

The present invention relates to a device and a method for determining a material parameter, in particular, for a plastic material or a process.

### BACKGROUND INFORMATION

At present, analyzing process or material characteristics relevant to this requires several measurements, some of which are time-intensive and cost-intensive, which means that an immediate decision regarding the state of the material and the process is not possible.

### SUMMARY

Below, a method and a device are described, by which process and/or material data may be acquired almost in real time. In this manner, processes may be optimized directly. This allows both uniform product quality to be ensured and costs for the material to be reduced. The present invention allows simple, precise and favorable product variations resulting from involuntary manipulation, e.g., batch fluctuations, or deliberate manipulations or changes, such as counterfeit products, to be detected in a timely manner. In addition, by intelligently linking the predicted material parameters and present process parameters, reliable and robust process windows may be set for optimum product quality.

In accordance with an example embodiment of the present invention, a method for determining a material parameter, in particular, for a plastic material or a process, provides for a combination of input variables to be supplied for a model, and for the material parameter to be determined as a function of the model; the model mapping the combinations of input variables to material parameters; the model being trained as a function of training data, which are defined by a plurality of combinations of input variables and their assignment to a setpoint material parameter; either the model continuing to be trained as a function of a result of a comparison of a material parameter determined by the model for one of the combinations from the training data, with the setpoint material parameter assigned to this combination in the training data; or a changed model being defined by adding a module to the model and/or by removing at least one module from the model, and the changed model being trained. This allows knowledge of a material, which may not be derived from the directly measurable chemical properties, to be acquired from the combinations.

The combination of input variables is preferably determined by spectral data, thermoanalytic method data, rheological data, data regarding melting viscosity, data about a diffraction method and/or chromatographic method; the model including a module, which determines the material parameter, using at least one classification and/or regression. These modules are particularly suitable.

The module preferably includes an artificial neural network (ANN) or a support vector machine (SVM), in particular, defined by partial least squares regression (PLS-Reg), partial least squares classification (PLS-DA), linear discriminant analysis (LDA), ridge regression, multiple linear regression (MLR), logistic regression, a decision or regression tree, a random forest. These methods are particularly suited for deriving the material parameter.

In one aspect of the present invention, a module for preprocessing the combination of input variables is provided, in particular, including detrending, derivation, mean centering, Savitzky-Golay filtering, Fourier transformation, standard normal variate (SNV). This improves the model further.

In one aspect of the present invention, a module is provided for eliminating disturbance from at least one of the input variables or their combination, in particular, using error removal by orthogonal subtraction (EROS), external parameter orthogonalization (EPO), wavelet transformation, or Fourier transformation. This renders the model more robust.

In one aspect of the present invention, a module is provided, which is configured for dimensionality reduction or feature selection, in particular, using principal component analysis (PCA), for dimensionality reduction, stepwise variable selection (SVS) or Procrustes variable selection. This allows the material parameter to be determined more efficiently.

In one aspect of the present invention, at least one module includes a classifier, which is configured to classify data in a class, which determines a manufacturer of a material, a group of manufacturers of a material, a material property, or a batch in which the material is manufactured. Consequently, chemical materials may be classified in a particularly simple manner.

In one aspect of the present invention, the input variables or their combination are classified consecutively by at least two classifiers. This cascading arrangement enables the individual classifiers to be constructed smaller and more efficiently.

In one aspect of the present invention, the input variables or their combination are classified consecutively by at least one artificial neural network and by at least one support vector machine. Consequently, the best possible arithmetic operation may be used as a function of the material parameter, which is intended to be determined ultimately.

In one aspect of the present invention, at least one module is configured for regression; the material parameter being determined by regression, in particular, a chemical composition, by which a type of polymer, an additive, a type of filler, a level of filler, a manufacturer, and/or a batch is clearly identifiable. This allows the manufacturer or the batch to be identified simply, for example, in a quality check.

As a function of at least one material parameter, at least one material property is preferably identified, and consequently, a difference from a setpoint value for it is discerned, or a setpoint value for a process window is set.

In accordance with an example embodiment of the present invention, a device for determining a material parameter, in particular, for a plastic material or a process, provides that the device include a plurality of processors, as well as at least one storage device for a model, which are configured to carry out the method.

Further advantageous specific embodiments of the present invention are derived from the following description and the figures.

### BRIEF DESCRIPTION OF THE DRAWINGS

FIG. 1 shows a device for determining a material parameter, in accordance with an example embodiment of the present invention.

FIG. 2 shows a method of determining a material parameter, in accordance with an example embodiment of the present invention.

FIG. 3 shows a classification model for determining the material parameter, in accordance with an example embodiment of the present invention.

### DETAILED DESCRIPTION OF EXAMPLE EMBODIMENTS

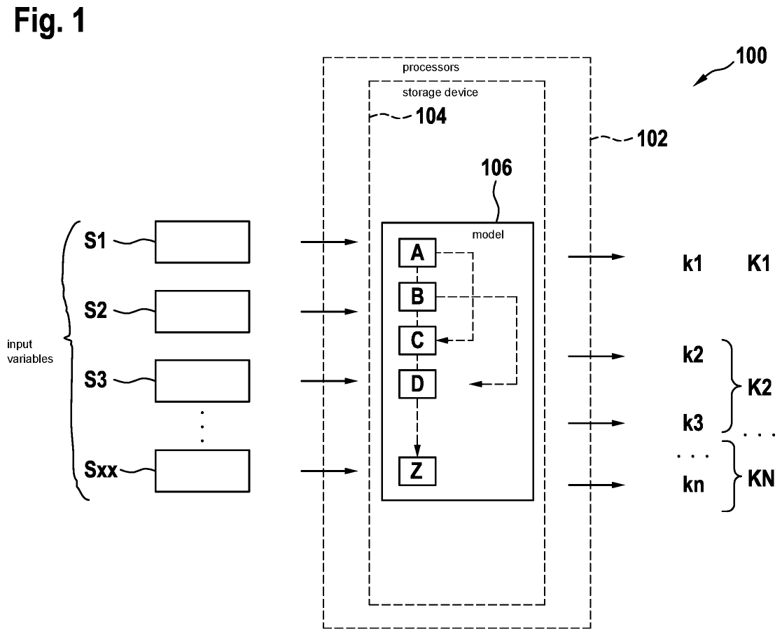

A device 100 for determining a material parameter of, in particular, a plastic is represented schematically in FIG. 1. In the example, a plurality of material parameters k1, k2, . . . , kn are determined from different categories K1, K2, . . . , KN. Device 100 includes a plurality of processors 102 and a storage device 104 for a model 106. Device 100 is configured to carry out the method described in the following. A powerful computer, which is configured to determine parameters of model 106, may be provided for, in particular, the training of model 106.

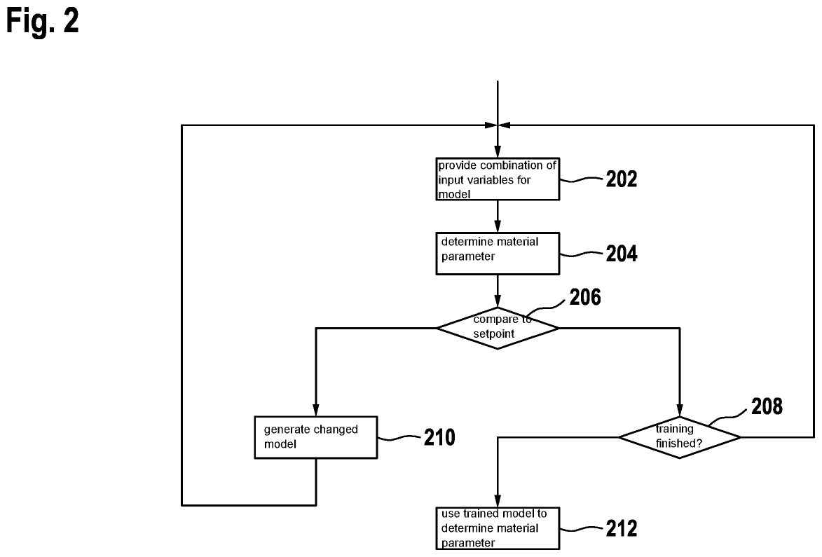

The method described below in light of FIG. 2 is used for determining a material parameter or a plurality of material parameters of a plastic or of a process. In the following, the determination of a material parameter of plastic, starting from input variables S1, . . . , Sxx, is described. The material parameter may characterize a chemical composition, a material property, a mechanical variable, or a process parameter. These may be in the following categories:

Category 1: Chemical Composition

type of polymer, compounding, additives, level of filler, polymer batch.

Category 2: Material Property

water content in the material (xH2O),
viscosity number, additive concentration, morphology, flowability, degree of cross-linking, viscosity of the material (e.g., shear viscosity/extensional viscosity), reactivity, coefficient of expansion, glass transition temperature.

Category 3: Mechanical Variables

elongation at fracture, breaking strength/tensile strength, modulus of elasticity, creep and relaxation processes.

Category 4: Category

Deviation (setpoint/actual), good/bad

At least one of the input variables S1, . . . , Sxx may characterize spectral data, thermoanalytic method data, rheological data, a melting viscosity, data regarding a diffraction method or a chromatographic method.

What is provided, is, in particular

spectral data (300 nm . . . 3 mm): UV vis., near-infrared (NIR), mid-infrared (FTIR), far-infrared (terahertz), Raman spectroscopy, chemiluminescence.

Thermoanalytic Method Data: thermogravimetry, differential thermal analysis (DSC), thermomechanical analysis, dynamic mechanical analysis.

Rheological method data: capillary rheometer and rotational rheometer, extensional rheometer.

Melting Viscosity Data: melt volume flow rate.

Data from Diffraction Methods: x-ray diffraction.

Chromatographic Method Data: gel permeation chromatography (GPC).

Input variables S1, . . . , Sxx may be sensorial data acquired by a sensor, or analytic data. These constitute the input variables of model 106 or its modules.

Model 106 contains at least one module, which may include a machine learning algorithm.

At least one module A may be provided, which is configured for preprocessing individual, or a plurality of the, input variables S1, . . . , Sxx or for preprocessing the combination of input variables S1, . . . , Sxx, in particular, using detrending, derivation, mean centering, Savitzky-Golay filtering, Fourier transformation, standard normal variate (SNV).

At least one module B may be provided, which is configured to eliminate disturbance from at least one of the input variables S1, . . . , Sxx or their combination, in particular, using error removal by orthogonal subtraction (EROS), external parameter orthogonalization (EPO), wavelet transformation, or Fourier transformation.

At least one module C may be provided, which is configured for dimensionality reduction or feature selection, in particular, using principal component analysis (PCA), for dimensionality reduction, stepwise variable selection (SVS) or Procrustes variable selection.

At least one module D may be provided, which reflects a classification and/or regression algorithm. Module D may be, for example, an artificial neural network or support vector machine. Classification and/or regression algorithms include, for example: partial least squares regression (PLS-Reg) or partial least squares classification (PLS-DA), linear discriminant analysis (LDA), ridge regression, multiple linear regression (MLR), logistic regression, decision and regression tree, random forest, support vector machine (SVM), artificial neural networks (ANN).

A further module Z or a plurality of further modules may also be provided, which implement functions specifiable by the user.

In a training phase, it is provided that model 106 be trained as a function of input data, which include data sets of input variables S1, . . . , Sxx and an assignment of each of the data sets to a setpoint parameter. Model 106 includes at least one module D, which is configured to determine the material parameter as a function of input variable S1, . . . , Sxx.

In a step 202, a combination of input variables S1, . . . , Sxx is provided for a model 106, and a setpoint material parameter assigned to this combination is supplied. Input variables S1, . . . , Sxx and the setpoint material parameter are retrieved from the training data during the training.

In a subsequent step 204, the material parameter is determined as a function of model 106 and as a function of the combination of input variables S1, . . . , Sxx.

In a step 206, in a comparison of a material parameter determined for the combination of input variables S1, . . . , Sxx and the setpoint material parameter assigned to this combination, a difference of this material parameter from the setpoint material parameter is determined as a result of the comparison. If the difference from the setpoint material parameter falls below a predefined difference, then a step 208 is carried out.

Otherwise, a step 210 is carried out.

In step 208, it is checked if the training is finished. If the training is finished, then a step 212 is carried out. Otherwise, step 202 is carried out. If step 202 is carried out anew, then the same model 106 continues to be trained.

In step 212, the model 106 trained in this manner is used for determining the material parameter. For example, the material parameter is determined for a plastic manufacturing process, a process in which plastic is used, or for a plastic material to be tested. For example, as a function of at least one material parameter, at least one material property is identified, and consequently, a difference from a setpoint value for it is discerned, or a setpoint value for a process window is set.

In step 210, a changed model is generated. The changed model may be generated by adding a module A, . . . , Z to the model. The changed model may be defined by removing at least one module A, . . . , Z from model 106. In this context, at least one of modules A, . . . , Z remains in the changed model. The addition may be carried out randomly and/or in an automated manner, or by an expert.

Subsequently, step 202 is carried out for the changed module. Consequently, the changed model is trained.

In order to classify thermoplastic synthetic materials, in one specific example, polyamide 66 is supplied, for example, in one variant, in the following combination particularly suitable for it:

Input Variables:

Spectral data in mid-infrared (FTIR), since in this case, a large number of information items about the molecular composition of the plastics are available, and due to the high dimensionality of the data, the use of ML algorithms is necessary.

Model:

Module A: Preprocessing: Fourier transformation for minimizing the signal noise, SNV transformation in order to eliminate an offset of the signals computationally.

Module B: Disturbance Elimination: EROS in order to eliminate variances, which do not come from the material parameter, and to attain a higher degree of robustness of the model.

Module C: Dimensional Reduction: PCA

Module D: Classification Algorithms: ANN and SMV, since these are suitable for nonlinear classification problems.

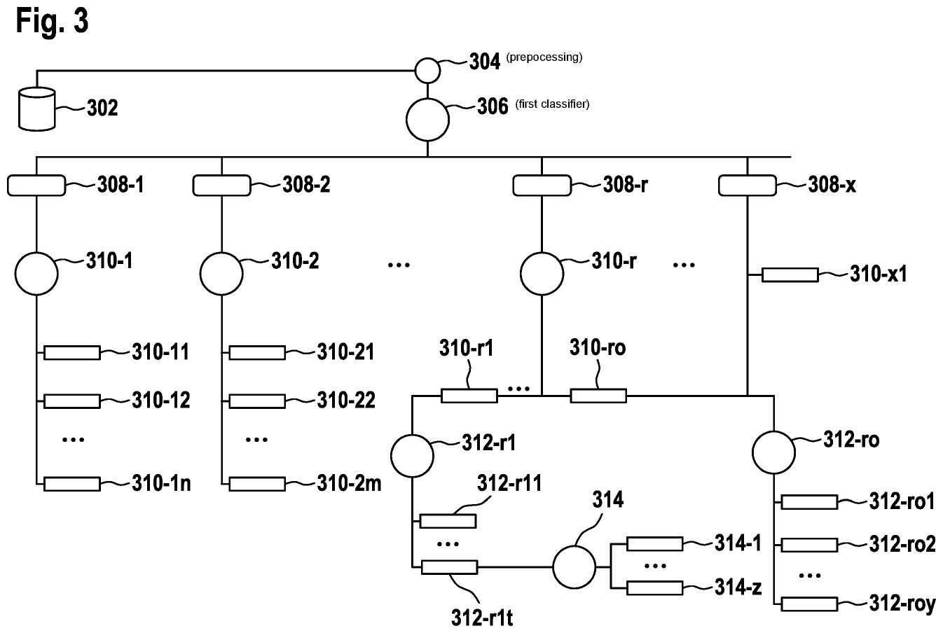

A classification model 300 is given in FIG. 3; with the aid of the classification model, polyamide 66 being able to be classified in a class, by which the material parameter is determined.

Input variables S1, . . . , Sxx are stored in a database 302 in the form of raw data. By way of preprocessing 304, the raw data arrive at a first classifier 306 in the form of preprocessed data.

Preprocessing 304 is implemented, for example, as one of modules A, B, C or a combination of these modules, or it may be omitted in other variants.

In the example, first classifier 306 is an artificial neural network having the following characteristics:

Input variable dimension: 600

1^{st} hidden layer: 600 neurons

Dropout 1^{st} layer: 70%

2^{nd} hidden layer: 60 neurons

Dropout 2^{nd} layer: 70%

Activation function: Softmax

In the example, first classifier 306 is trained in 25 epochs as a function of the training data.

First classifier 306 classifies the preprocessed data in a class from a number x of classes 308-1, . . . , 308-x, which, in the example, characterize a specific manufacturer of polyamide 66. As shown in the example with the aid of class 308-r, a group of manufacturers may also be combined into one class. If an assignment to one of the manufacturers already determines polyamide 66 unequivocally, then the classification is ended. This is shown in the example of class 308-x, according to which polyamide 66 is classified in a class denoted by 310-x1 in FIG. 3.

If the first classifier classifies the data in a class, which determines a manufacturer unequivocally, the classified, preprocessed data may be used for a manufacturer-specific classification.

For example, the classified, preprocessed data for a first manufacturer, which is defined by a class denoted by 308-1 in FIG. 3, is reclassified by a second classifier 310-1 in a class from a number n of classes 310-11, 310-12, . . . , 310-1n. If polyamide 66 is determined unequivocally by an assignment to one of these classes, then the classification is ended. This is represented in the example of classes 310-11, 310-12, . . . , 310-1n.

In the example, second classifier 310-1 is an artificial neural network having the following characteristics:

Input variable dimension: 600

1^{st} hidden layer: 600 neurons

Dropout 1^{st} layer: 70%

2^{nd} hidden layer: 60 neurons

Dropout 2^{nd} layer: 70%

Activation function: Softmax

In the example, second classifier 310-1 is trained in 25 epochs as a function of the training data.

For example, the classified, preprocessed data of a first manufacturer, which is defined by a class denoted by 308-1 in

FIG. 3, is reclassified by a third classifier 312-1 in a class from a number m of classes 312-11, 312-12, . . . , 312-1m. If polyamide 66 is determined unequivocally by an assignment to one of these classes, then the classification is ended. This is represented in the example for classes 312-11, 312-12, . . . , 312-1m.

In the example, third classifier 312-2 is a support vector machine having the following characteristics:

Input variable dimension: 600

Kernel: RBF

Penalty Parameter C: 280

Gamma: 0.0017

In the example, third classifier 312-2 is trained as a function of the training data, until a maximum number of iterations or convergence is reached.

In the example, a fourth classifier 312-r is used for class 310-r, which combines a group of manufacturers.

For example, the classified, preprocessed data for the group of manufacturers are reclassified by fourth classifier 310-r in a class from a number o of classes 310-r1, . . . , 310-ro. In the example, a manufacturer from the group of manufacturers is determined by the assignment to one of these classes.

In the example, fourth classifier 312-r is an artificial neural network having the following characteristics:

Input variable dimension: 600

1^{st} hidden layer: 600 neurons

Dropout 1^{st} layer: 80%

2^{nd} hidden layer: 60 neurons

Dropout 2^{nd} layer: 80%

Activation function: Softmax

In the example, fourth classifier 312-r is trained in 40 epochs as a function of the training data.

If, on the basis of its manufacturer, polyamide 66 is determined unequivocally by an assignment to one of these classes, then the classification is ended. This is not represented in the example. In the example, a fifth classifier 312-r1 is used for one of the manufacturers from the group of manufacturers, and a sixth classifier 312-ro is used for another of the manufacturers of the group of manufacturers.

Fifth classifier 312-r1 classifies the data assigned to the one of the manufacturers of the group in a class from a number t of classes 312-r11, . . . , 312-r1t. In the example, fifth classifier 312-r1 is an artificial neural network having the following characteristics:

Input variable dimension: 600

1^{st} hidden layer: 600 neurons

Dropout 1^{st} layer: 70%

2^{nd} hidden layer: 60 neurons

Dropout 2^{nd} layer: 70%

Activation function: Softmax

In the example, fifth classifier 312-r1 is trained in 25 epochs as a function of the training data.

If polyamide 66 is determined unequivocally by an assignment to one of these classes, then the classification is ended. This is shown in the example for the class denoted by 312-r11 in FIG. 3. A seventh classifier 314 is provided for another class denoted by 312-r1t in FIG. 3. This is configured, for example, to classify the data in a class from a number z of classes 314-1, . . . , 314-z; in the example, the data characterizing a batch of polyamide 66.

In the example, seventh classifier 314 is an artificial neural network having the following characteristics:

Input variable dimension: 600

1^{st} hidden layer: 600 neurons

Dropout 1^{st} layer: 70%

2^{nd} hidden layer: 60 neurons

Dropout 2^{nd} layer: 70%

Activation function: Softmax

In the example, fifth classifier 312-r1 is trained in 25 epochs as a function of the training data.

A class, in which data characterizing an unknown polyamide or an unknown batch are classified, may be provided for each of the classifiers.

Sixth classifier 312-ro classifies the data assigned to the one of the manufacturers of the group in a class from a number y of classes 312-ro1, . . . , 312-roy. In the example, fifth classifier 312-ro is an artificial neural network having the following characteristics:

Input variable dimension: 600

1^{st} hidden layer: 600 neurons

Dropout 1^{st} layer: 70%

2^{nd} hidden layer: 60 neurons

Dropout 2^{nd} layer: 70%

Activation function: Softmax

In the example, sixth classifier 312-ro is trained in 25 epochs as a function of the training data.

If polyamide 66 is determined unequivocally by an assignment to one of these classes, then the classification is ended. This is shown in the example for the classes denoted by 312-ro1 to 312-roy in FIG. 3.

A further variant may provide a regression of a material property, in the example, a moisture content of a thermoplastic synthetic material, for example, polyamide 66.

The following combination is suitable for that.

Input Variables:

Spectral data in mid-infrared (FTIR), since in this case, a large number of information items about the molecular composition of the plastics are available, and due to the high dimensionality of the data, the use of ML algorithms is necessary. In addition, the FTIR spectroscopy is highly sensitive for determining the water content.

Model:

Module A: Preprocessing: Savitzky-Golay filtering for minimizing the noise, SNV transformation in order to eliminate an offset of the signals computationally.

Module B: Disturbance Elimination: EROS, in order to eliminate variances, which do not come from the material parameter, and to attain a higher degree of robustness of the models.

Module C: Feature Selection: stepwise variable selection: in order to select the variables, which correlate the most with the material parameter and increase the predictive accuracy of the model.

Module D: Regression Algorithms: partial least squares regression, since these linear relationships may be processed effectively in a multidimensional data space and have little tendency towards overfitting.

Using this model, the material parameter may be determined by regression. In the example, a chemical composition is determined, by which a type of polymer, an additive, a type of filler, a level of filler, a manufacturer, and/or a batch is clearly identifiable.

The method or the device, in which the method is implemented, may be used in an area of plastic processing, for example, for inspection of deliveries, for quality control in the production, and for analyzing field returns.

1-14. (canceled)

15. A method of determining a material parameter for a plastic material or a process, the method comprising the following steps:
providing a combination of input variables for a model;
determining the material parameter as a function of the model, the model mapping different combinations of input variables to material parameters;
training the model as a function of training data, which are defined by a plurality of combinations of the input variable and a respective assignment of each combination of the plurality of combinations from the training data to a setpoint material parameter; and
as a function of a result of a comparison of a respective material parameter determined by the model for one of the combinations from the training data, with the setpoint material parameter assigned to the one of the combinations from the training data, either: (i) continuing to train the model, or (ii) defining a changed model by adding a module to the model and/or by removing at least one module from the model and training the changed model.

16. The method as recited in claim 15, wherein each of the combinations of input variables is determined by spectral data, an/or thermoanalytic method data, and/or rheological data, and/or data regarding a melting viscosity, and/or data about a diffraction method and/or a chromatographic method, and wherein the model includes a module which determines the material parameter, using at least one classification and/or one regression.

17. The method as recited in claim 16, wherein the module includes an artificial neural network or a support vector machine, the module being defined by partial least squares regression partial least squares classification, and/or linear discriminant analysis, and/or ridge regression, and/or multiple linear regression, and/or logistic regression, and/or a decision or regression tree, and/or a random forest, and/or a support vector machine, and/or at least one artificial neural network.

18. The method as recited in claim 15, wherein the model includes a module for preprocessing the combination of input variables using detrending, and/or derivation, and/or mean centering, and/or Savitzky-Golay filtering, and/or Fourier transformation, and/or standard normal variate.

19. The method as recited in claim 15, wherein the model includes a module configured to eliminate disturbance from at least one of the input variables or their combination, using error removal by orthogonal subtraction or external parameter orthogonalization or wavelet transformation or Fourier transformation.

20. The method as recited in claim 15, wherein the model includes a module configured for dimensionality reduction or feature selection, using principal component analysis for dimensionality reduction, or stepwise variable selection, or Procrustes variable selection.

21. The method as recited in claim 15, wherein the model includes at least one module including a classifier, which is configured to classify data in a class, which determines a manufacturer of a material, or a group of manufacturers of the material, or a material property, or a batch, in which the material is manufactured.

22. The method as recited in claim 21, wherein the input variables or a combination pf the input variables are classified consecutively by at least two classifiers.

23. The method as recited in claim 21, wherein the input variables or a combination of the input variables are classified consecutively by at least one artificial neural network and by at least one support vector machine.

24. The method as recited in claim 15, wherein the model includes at least one module configured for regression, the material parameter being determined by regression, the material property being a chemical composition, by which a type of polymer, and/or an additive, and/or a type of filler, and/or a level of filler, and/or a manufacturer, and/or a batch is identifiable.

25. The method as recited in claim 15, wherein at least one material property is identified as a function of at least one material parameter, and a difference from a setpoint value for the at least one property is discerned, or a setpoint value for a process window is set.

26. A device for determining a material parameter for a plastic material or a process, the device comprising:
a plurality of processors; and
at least one storage device for a model;
wherein the device is configured to:
provide a combination of input variables for the model,
determine the material parameter as a function of the model, the model mapping different combinations of input variables to material parameters;
train the model as a function of training data, which are defined by a plurality of combinations of the input variable and a respective assignment of each combination of the plurality of combinations from the training data to a setpoint material parameter; and
as a function of a result of a comparison of a respective material parameter determined by the model for one of the combinations from the training data, with the setpoint material parameter assigned to the one of the combinations from the training data, either: (i) continue to train the model, or (ii) define a changed model by adding a module to the model and/or by removing at least one module from the model and train the changed model.

27. A non-transitory machine-readable storage medium on which is stored a computer program for determining a material parameter for a plastic material or a process, the computer program, when executed by a computer, causing the computer to perform the following steps:
providing a combination of input variables for a model;
determining the material parameter as a function of the model, the model mapping different combinations of input variables to material parameters;
training the model as a function of training data, which are defined by a plurality of combinations of the input variable and a respective assignment of each combination of the plurality of combinations from the training data to a setpoint material parameter; and
as a function of a result of a comparison of a respective material parameter determined by the model for one of the combinations from the training data, with the setpoint material parameter assigned to the one of the combinations from the training data, either: (i) continuing to train the model, or (ii) defining a changed model by adding a module to the model and/or by removing at least one module from the model and training the changed model.
